(19) United States
(12) Patent Application Publication
(10) Pub. No.: US 2019/0350584 A1
(43) Pub. Date: Nov. 21, 2019

(54) SUTURING INSTRUMENT CARTRIDGE WITH SUTURE PATH HAVING NEEDLE BARRIERS

(71) Applicant: Ethicon LLC, Guaynabo, PR (US)

(72) Inventor: Kevin M. Montgomery, Morrow, OH (US)

(21) Appl. No.: 15/979,707

(22) Filed: May 15, 2018

(51) Int. Cl. A61B 17/06; A61B 17/04

(52) U.S. Cl. CPC A61B 17/06133; A61B 17/0482; A61B 17/0483; A61B 17/0485; A61B 2017/06076; A61B 17/06066; A61B 17/0491; A61B 2017/06142; A61B 17/0493

(57) ABSTRACT

A needle cartridge for a surgical suturing instrument includes a cartridge body, a track extending through the cartridge body and defining a circular path, an arcuate needle movably positioned within the track, and a suture thread extending from the arcuate needle. A needle driver operatively connects to the cartridge body and the arcuate needle. The needle driver is configured to orbit the arcuate needle along the circular path. A needle cover is secured to the cartridge body such that the needle cover and the cartridge body define a gap, which receives the suture thread therethrough. A retention abutment extends from at least one of the cartridge body or the needle cover into the gap such that the needle cover, the cartridge body, and the retention abutment at least partially define a tortuous path configured to retain the orbiting arcuate needle in the track while the suture thread passes therethrough.

### BACKGROUND

Sutures may be used in a wide variety of surgical procedures. Manual suturing may be accomplished by the surgeon using a fine pair of graspers to grab and hold a suture needle, pierce the tissue with the needle, let go of the needle, and re-grasp the needle to pull the needle and accompanying suture thread through the tissues to be sutured. Such needles may be curved with the suture attached to the trailing end of the needle.

Some surgical instruments automate at least part of the suturing procedure. Examples of automated suturing instruments are described in U.S. Pat. No. 8,702,732, entitled “Laparoscopic Suturing Instrument with Dual-Action Needle Graspers,” issued Apr. 22, 2014, the disclosure of which is incorporated by reference herein; U.S. Pat. No. 9,168,037, entitled “Laparoscopic Suture Device with Asynchronous In-Line Needle Movement,” issued Oct. 27, 2015, the disclosure of which is incorporated by reference herein; U.S. Pat. No. 9,357,998, entitled “Circular Needle Applier with Articulating and Rotating Shaft,” issued Jun. 7, 2016, the disclosure of which is incorporated by reference herein; and U.S. Pat. No. 9,474,522, entitled “Jawed Cartridge Receiving Assembly for Needle Cartridge,” issued Oct. 25, 2016, the disclosure of which is incorporated by reference herein.

While various kinds of suturing instruments and associated components have been made and used, it is believed that no one prior to the inventor(s) has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a side view of an exemplary surgical suturing instrument;

FIG. 2A depicts top perspective exploded view of a cartridge receiving assembly of the instrument of FIG. 1;

FIG. 2B depicts bottom perspective exploded view of the cartridge receiving assembly of FIG. 2A;

FIG. 3A depicts a top perspective view of a first exemplary needle cartridge configured for receipt in the needle cartridge receiving assembly of FIG. 2A;

FIG. 3B depicts a bottom perspective view of the needle cartridge of FIG. 3A;

FIG. 4 depicts an exploded view of the needle cartridge of FIG. 3A;

FIG. 5A depicts a perspective view of a drive assembly of the needle cartridge of FIG. 3A, with the drive assembly at one end of its stroke;

FIG. 5B depicts a perspective view of the drive assembly of FIG. 5A, with the drive assembly at mid-stroke;

FIG. 5C depicts a perspective view of the drive assembly of FIG. 5A, with the drive assembly at the other end of its stroke;

FIG. 6 depicts a partial plan view of a needle driver of the needle cartridge of FIG. 3A engaging a needle of the needle cartridge of FIG. 3A;

FIG. 7 depicts a right front perspective view of a second exemplary needle cartridge;

FIG. 8 depicts a left front partially exploded perspective view of the needle cartridge of FIG. 7;

FIG. 9 depicts an upper right front perspective view of the needle cover of FIG. 8;

FIG. 10 depicts a lower left front perspective view of the needle cover of FIG. 9;

FIG. 11 depicts an enlarged, left front perspective view of the upper body of the cartridge body of FIG. 8;

FIG. 12 depicts an enlarged, right front perspective view of the upper body of the cartridge body of FIG. 11;

FIG. 13 is an enlarged, perspective, sectional view the needle cartridge of FIG. 7 taken along section line 13-13 of FIG. 7;

FIG. 14 depicts an enlarged, top view of the upper body of the cartridge body of FIG. 11;

FIG. 15 depicts an enlarged, bottom view of the needle cover FIG. 9;

FIG. 16A depicts an enlarged, front perspective view of the needle cartridge of FIG. 7 showing the arcuate needle in a first position;

FIG. 16B depicts the enlarged, front perspective view of the needle cartridge similar to FIG. 16A, but showing the arcuate needle orbiting from the first position toward a second position;

FIG. 16C depicts the enlarged, front perspective view of the needle cartridge similar to FIG. 16B, but showing the arcuate needle orbiting from the second position toward a third position; and

FIG. 16D depicts the enlarged, front perspective view of the needle cartridge similar to FIG. 16C, but showing the arcuate needle orbiting from the third position toward a fourth position.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown. DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

For clarity of disclosure, the terms “proximal”, “distal”, “upper”, “lower”, “top”, and “bottom” are defined herein relative to an operator or other operator grasping a surgical instrument having a distal surgical end effector. The term “proximal” refers the position of an element closer to the operator or other operator, and the term “distal” refers to the position of an element closer to the surgical end effector of the surgical instrument and further away from the operator or other operator. The terms “top” and “upper” refer to the position of the element closer to a top of the surgical instrument when viewed by the operator from above, and the terms “bottom” and “lower” refersto the position of the element closer to a bottom of the surgical instrument when viewed by the operator from below. As such, proximal and distal portions are generally in longitudinal opposition as described herein, whereas upper and lower portions are generally in transverse opposition as described herein. The term “lateral” is also used herein to describe the lateral direction, which is perpendicular to the transverse direction. Such terms are used below with reference to views as illustrated for clarity and are not intended to limit the invention described herein.

### I. Overview of Exemplary Surgical Suturing Instrument

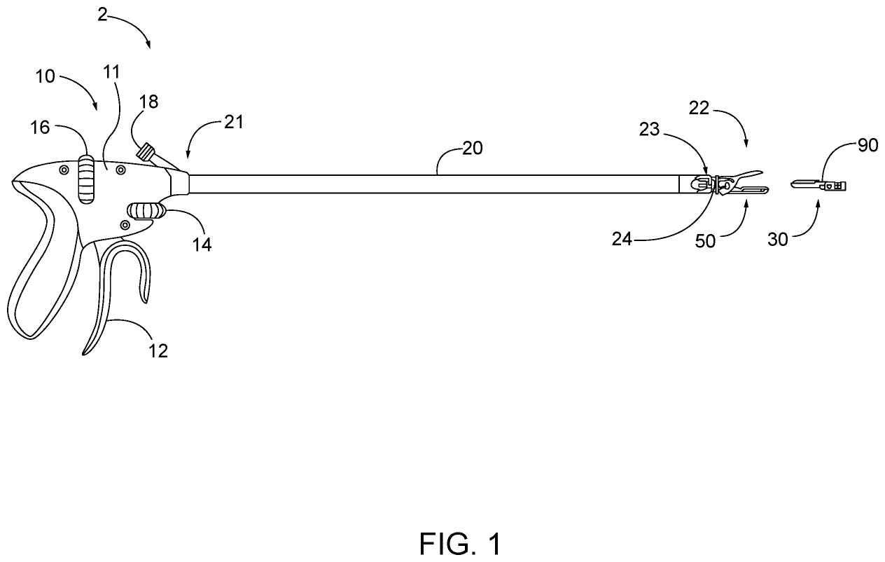

FIG. 1 illustrates an example of a surgical suturing instrument (2). Instrument (2) comprises a handle assembly (10) and a shaft assembly (19) having an elongate shaft (20) extending from a proximal end portion (21) to a distal end portion (22) thereof. Distal end portion (22) includes a cartridge receiving assembly (50), which is operable to receive a first exemplary needle applier cartridge (30), which may also be referred to herein as a needle cartridge (30). Shaft (20) defines a longitudinal axis extending from proximal end portion (21) to distal end portion (22). Handle assembly (10) is connected to proximal end portion (21) of shaft (20). In this example handle assembly (10) is a manual pistol grip handle. However, a variety of other manual actuators could also be used, including but not limited to a scissor grip handle, a syringe grip handle, endoscopic rotary knobs, and the like. Handle assembly (10) could also take the form of a robotic interface, such as a DAVINCI puck, or a housing comprising gears or pulleys, servomechanisms, and the like.

Needle applier cartridge (30) is connected to distal end portion (22) of shaft (20) via cartridge receiving assembly (50). Needle applier cartridge (30) is operable to rotate an arcuate needle in a circular path enabling a surgeon to selectively apply sutures. In some alternative versions, needle applier cartridge (30) is integral with shaft (20) and handle assembly (10) as a unitary disposable instrument intended for a single surgical procedure. Needle applier cartridge (30) may also be integral with shaft (20) and handle assembly (10) as a reusable instrument. Optionally, as illustrated here, needle applier cartridge (30) may be provided in a disposable cartridge body (90) and shaft (20) includes cartridge receiving assembly (50) to releasably hold cartridge body (90). In some such versions, shaft (20) and handle assembly (10) may also be disposable or reusable. Versions with reusable components are intended to be cleaned, sterilized, and reused for multiple surgical procedures, and may include a flush port (18) to facilitate cleaning. The preferable life cycle of a reusable instrument is at least 50 operations, more preferably at least 150 operations, and most preferably at least 200 operations. Reusable components may be built using materials that can withstand autoclave sterilization temperatures of at least 135 degrees Celsius, although low temperature materials can also be used with low temperature sterilization techniques known in the art.

A first user input member (12), shown here as a trigger, pivots between opened and closed positions, may be used to selectively actuate needle applier cartridge (30). The trigger may be spring biased to return the trigger to its open position. A second user input member (14), shown here as a rotary knob, may be used to selectively articulate shaft (20). A third user input member (16), shown here as a rotary knob, may be used to selectively rotate needle applier cartridge (30) about shaft (20). Of course, the number, type, configuration, and operation of first, second, and third input members (12, 14, 16) may vary.

Shaft (20) includes an articulation joint (23). Second user input feature (14) is operable to selectively articulate joint (23) via a joint drive assembly (not shown). Rotary knob (14) rotates in a plane spaced below and generally parallel with shaft (20). An axle (not shown) connects second user input feature (14) to a disk (not shown) in housing (11) that also rotates in a plane generally parallel with the shaft (20) to position distal end portion (22) of shaft assembly (19) relative to proximal end portion (21).

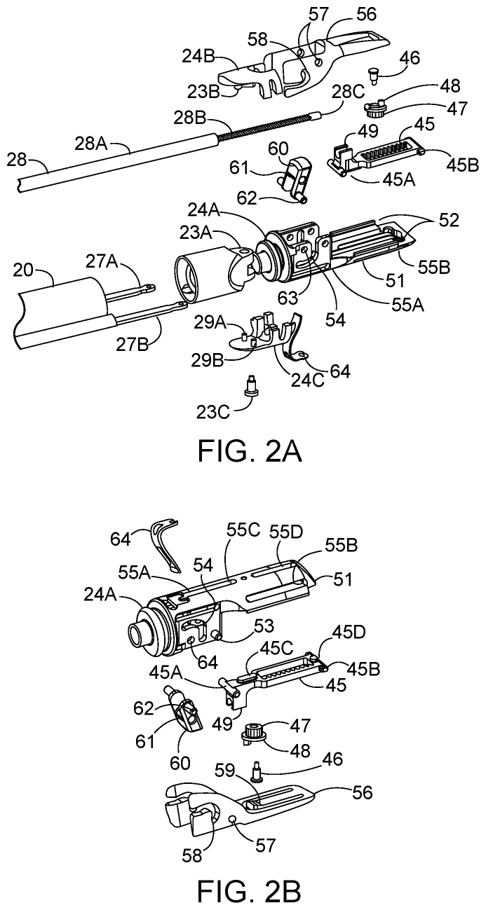

FIGS. 2A-2B illustrate exploded views of cartridge receiving assembly (50) of the present example. Distal end portion (22) of shaft (20) comprises articulation joint (23) and a rotational bearing (24). Articulation joint (23) includes a knuckle (23A) that receives pins (23B, 23C), which are connected to bearing supports (24B, 24C). Thus, pins (23B, 23C) define the pivoting axis for articulation joint (23), enabling cartridge receiving assembly (50) to articulate left and right relative the shaft (20), away from the longitudinal axis defined by shaft (20). Rods (27A, 27B) are operably connected to articulation joint (23). In this example, rods (27A, 27B) extend through shaft (20), through knuckle (23A), and connect to pins (29A, 29B) on bearing support (24C). Rods (27A, 27B) are operatively connected to rotary knob (14) to opposingly push and pull rods (27A, 27B). In other words, second user input feature (14) (14) is operable to drive rods (27A, 27B) at the same time in opposite longitudinal directions, such that rod (27A) will translate distally while rod (27B) translates proximally; and such that rod (27B) will translate distally while rod (27A) translates proximally. Because pins (29A, 29B) are laterally spaced from the pivoting axis, the simultaneous push and pull action will in turn articulate cartridge receiving assembly (50) about joint (23) relative to shaft (20).

Rotational bearing (24) is positioned distal relative to articulation joint (23). Bearing (24) includes a circumferential flange (24A) that is captured between the bearing supports (24B, 24C) such that the flange (24A) can rotate relative the bearing supports (24B, 24C) and enabling unbounded rotation of cartridge receiving assembly (50) relative shaft (20) about the longitudinal axis defined by shaft (20). A drive rod (28) extends through shaft (20). In this example, drive rod (28) comprises a proximal rigid portion (28A) and a distal bendable portion (28B) that are fixedly connected to one another. Bendable portion (28B) extends through articulation joint (23) and through bearing (24); distal end (28C) is fixedly connected to a mount (49) on a rack (45).

Cartridge receiving assembly (50) includes a transmission mechanism (44) configured to transfer force from input trigger (12) to cartridge (30) for actuation thereof. Transmission mechanism (44) includes rack (45) reciprocates longitudinally in lower jaw (51) with followers (45A, 45B, 45C, 45D) constrained in tracks (55A, 55B, 55C, 55D), respectively. Tracks (55A, 55B, 55C, 55D) open through lower jaw (51), providing fluid passages to the internal components within the lower jaw (51), thus facilitating easier cleaning. A pinion (47) is mounted to lower jaw (51) by a pin (46) in the rack (45) such that longitudinal reciprocation of the rack (45) is converted into rotational reciprocation of pinion (47). A key (48) of transmission mechanism (44) communicates the reciprocating rotation to a rotary input (94) in cartridge body (90), which in turn actuates needle applier cartridge (30).

Drive rod (28) is operatively connected to first user input member (12) and to third user input member (16). Actuation of first user input member (12) will impart axial push and pull loads on drive rod (28) to longitudinally reciprocate rack (45) and thereby actuate needle applier cartridge (30). Actuation of third user input member (16) will impart a rotational load on drive rod (28) thus rotating cartridge receiving assembly (50) about bearing (24) relative to shaft (20). Accordingly, a single drive rod (28) operates to both actuate needle applier cartridge (30) as well as control distal rotation of needle applier cartridge (30) about the longitudinal axis of shaft (20). By consolidating dual functions with a single drive rod (28), the number of components is reduced, and more space is provided in the shaft (20), which may make the device less expensive to manufacture and easier to clean.

Cartridge receiving assembly (50) is dimensioned and adapted to receive and hold cartridge body (90). As shown in FIGS. 2A-2B, cartridge receiving assembly (50) of this example has upper and lower jaws (56, 51) that are operable to transition between an open configuration and a closed configuration. In the closed configuration, jaws (56, 51) are operable to receive and retain cartridge body (90). In the closed configuration, jaws (56, 51) are operable to release cartridge body (90). In the present example, lower jaw (51) is stationary and upper jaw (56) pivots. Alternatively, the arrangement could be reversed, or in some versions both jaws (56, 51) could pivot. Lower jaw (51) has two laterally offset longitudinal rails (52) that are dimensioned and adapted to receive cartridge body (90). Rails (52) help longitudinally align cartridge body (90) in cartridge receiving assembly (50) and laterally retain cartridge body (90) in jaws (51, 56). Upper jaw (56) pivots relative lower jaw (51) about a pin (53) that is received in holes (57). A tooth (59) is resiliently oriented downwardly from upper jaw (56) toward lower jaw (51) with a ramped distal face and a stepped proximal face. Tooth (59) is dimensioned and adapted to latch with cartridge body (90) and longitudinally retain cartridge body (90) in jaws (51, 56). Tooth (59) deflects by virtue of a resilient cantilevered arm extending proximally from the distal end of upper jaw (56). In this example, tooth (59) and the cantilevered arm are monolithic with upper jaw (56), thus reducing the number of components and moving pieces, which may make the device less expensive to manufacture and easier to clean.

A button (60) is operable to open and close jaws (51, 56). While button (60) could be placed on or near the handle assembly (10) in some versions, in this example button (60) is positioned adjacent cartridge receiving assembly (50), which eliminates a linkage in shaft (20) thus creating space in shaft (20) and making the device less expensive and easier to clean. The action of button (60) may vary, but in this example button (60) pivots relative to lower jaw (51) about a pin (63) that is received in a hole (61). A follower (62) is received by cam slots (54, 58). Pivoting button (60) proximally will open jaws (51, 56), while pivoting button (60) distally will close jaws (51, 56). A spring (64) engages and biases button (60) distally. By pulling button (60) proximally, follower (62) will drive cam slot (58) to open upper jaw (56). When button (60) is released, spring (64) will resiliently drive button (60) distally to close upper jaw (56).

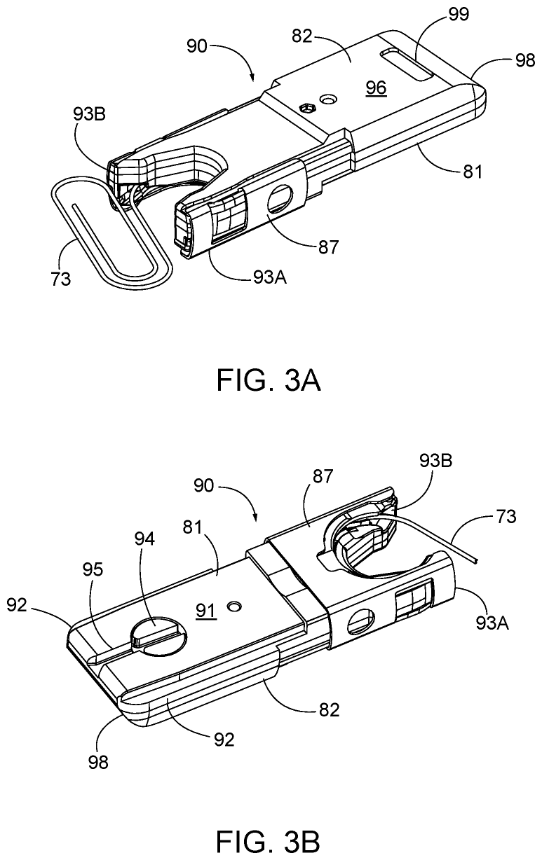

FIGS. 3A-3B illustrate cartridge body (90) of the present example in greater detail. A lower face (91) of cartridge body (90) is adapted to engage lower jaw (51); and an upper face (96) is adapted to engage upper jaw (56). Poke-yoke features on cartridge body (90) prevent improper insertion of cartridge body (90) into cartridge receiving assembly (50), but also contribute to the aesthetic appearance of cartridge body (90). For instance, lower face (91) has a pair of longitudinal notched shoulders (92) that are dimensioned to interface and mate with rails (52). In this example, notched shoulders (92) are shaped as a stepped rabbet, but a variety of other aesthetic shapes could also be employed such as chamfers and radii. In contrast, upper face (96) is asymmetrical relative lower face (91) and lacks shoulder notches, so upper face (96) would interfere with rails (52) if cartridge body (90) were inserted upside-down in cartridge receiving assembly (50). In another instance, the geometry of a proximal face (98) of cartridge body (90) is vertically asymmetrical and thus prevents cartridge body (90) from being inserted upside-down between jaws (51, 56). In this example, proximal face (98) comprises a curved surface that gently transitions to upper face (96), which matches similar geometry in cartridge receiving assembly (50); while the transition to lower face (91) has a tighter radius. Of course, a variety of other asymmetrical aesthetic geometries could also be employed that could contribute to the visual appearance and/or poke-yoke aspects of cartridge body (90).

Arms (93A, 93B) define a generally U-shaped distal end on cartridge body (90). A slot (95) and rotary input (94) are aligned and dimensioned to receive the key (48) while cartridge body (90) is being slid into cartridge receiving assembly (50). When cartridge body (90) is fully seated into cartridge receiving assembly (50), a step (99) aligns with and receives tooth (59) to latch cartridge body (90) in cartridge receiving assembly (50). Key (48) also aligns with rotary input (94), thereby providing a torsional interface that rotationally couples pinion (47) and rotary input (94). In use, the needle (70) exits arm (93A) and enters arm (93B).

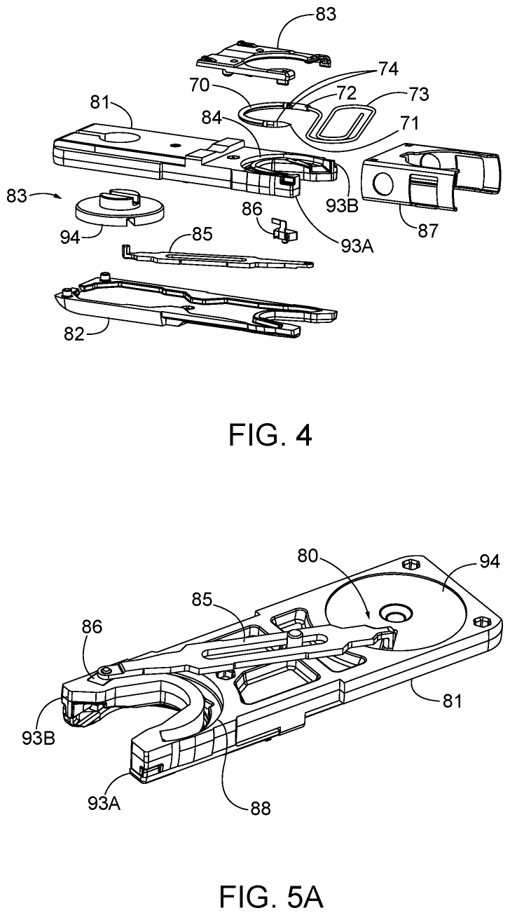

As shown in FIGS. 3A-4, cartridge body (90) further comprises a lower body (81), an upper body (82), a needle (70), a needle cover (83) and a drive assembly (80) configured to drive needle (70). Drive assembly (80) includes a needle driver (86), rotary input (94), and a link (85) are captured between lower body (81) and upper body (82). Bodies (81, 82) may be attached to one another using a variety of known techniques, including welds, pins, adhesives, and the like to form cartridge body (90). Needle (70) has a leading end (71) and a length of suture (73) extending from a trailing end (72) thereof. Needle (70) orbits in a circular path defined by a needle track (84) and between arms (93A, 93B). Needle (70) includes notches (74) that are configured to facilitate engagement between needle driver (86) and needle (70). Needle (70) is captured in needle track (84) by needle cover (83). A cage (87) slides over bodies (81, 82) and needle cover (83) to attach needle cover (83) against lower body (81).

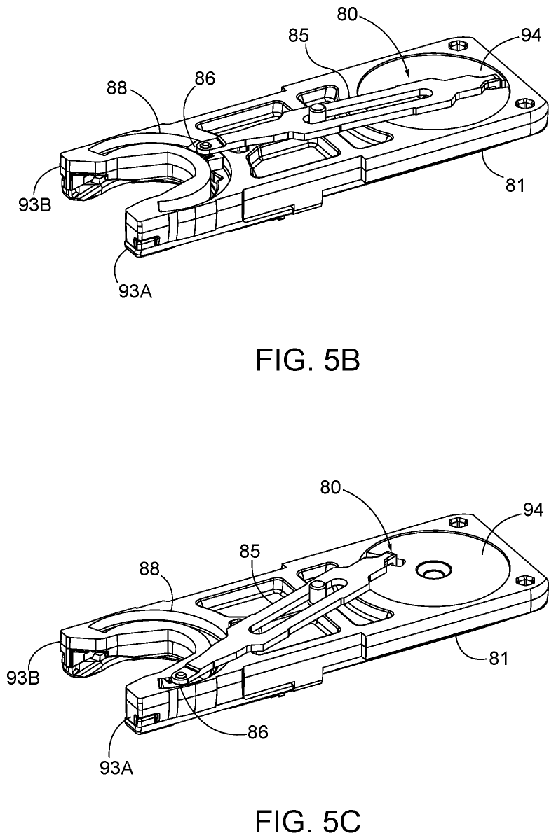

FIGS. 5A-5C illustrate an example of a drive stroke of the transmission in cartridge body (90) for driving needle (70) in a circular, orbital path. However, it should be understood that needle (70) and suture (73) are omitted from FIGS. 5B-5C for clarity. Needle driver (86) rides in a carrier track (88) and extends into needle track (84) (see FIG. 4) to engage and drive needle (70). Link (85) connects rotary input (94) to needle driver (86). FIG. 5A shows needle driver (86) positioned at one end of its stroke in carrier track (88). As shown in FIG. 5B, counterclockwise rotation of rotary input (94) will translate needle driver (86) clockwise along carrier track (88), thereby driving needle (70) clockwise. As shown in FIG. 5C, continued counterclockwise rotation of the rotary input (94) will continue to translate needle driver (86) and thereby drive needle (70) clockwise until it reaches the other end of its stroke in carrier track (88). In this example, the drive stroke rotates the needle (70) in its circular path along an angular range of about 180 degrees. For the return stroke, the sequence can be reversed by rotating the rotary input (94) clockwise, which will translate needle driver (86) counterclockwise in carrier track (88). Needle driver (86) is disengaged from needle (70) during the return stroke until needle driver (86) reaches the end of the return stroke. Needle driver (86) will re-engage needle (86) upon completing the return stroke. Thus, a sequence of drive and return strokes will rotate the needle (70) in a circular path.

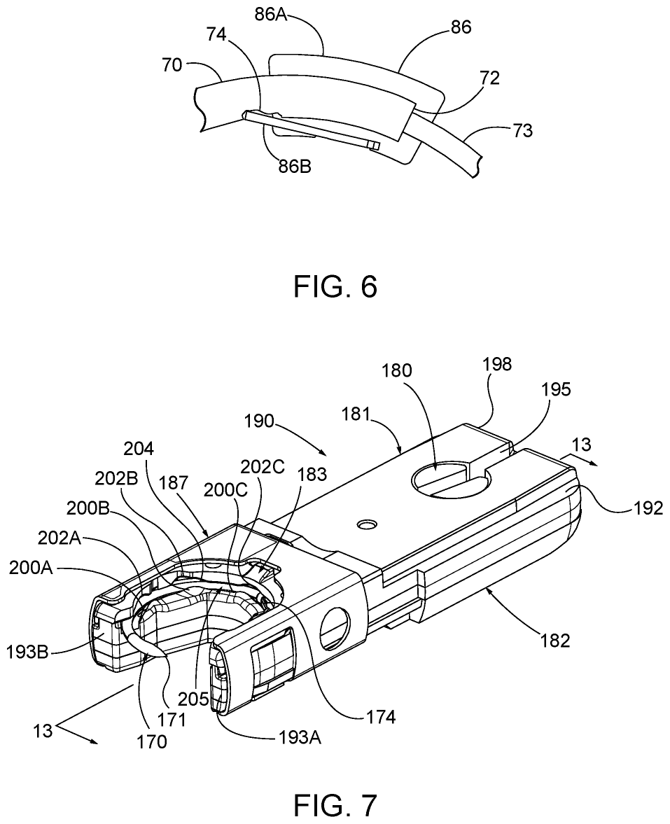

FIG. 6 shows a detailed view of needle driver (86) engaging needle (70). Needle driver (86) comprises a carrier (86A) and a driver (86B). Carrier (86A) is dimensioned to slidably fit in carrier track (88). Driver (86B) is attached to carrier (86A) and is operative to engage needle (70) at an oblique angle. Leftward movement of needle driver (86) will cause driver (86B) to engage proximal notch (74) of needle (70) during the drive stroke. When so engaged, needle (70) will slide in needle track (84) in unison with needle driver (86). Due to the oblique angle, rightward movement of needle driver (86) will disengage driver (86B) from proximal notch (74) of needle (70) and slide over the stationary needle (70) during the return stroke.

Referring back to FIGS. 5A-5C and FIG. 6, when first user input member (12) (see FIG. 1) is depressed, closing the trigger, needle driver (86) will be actuated through its drive stroke where it orbits along an angular range of motion at least about 180 degrees counterclockwise to a driven position as shown in FIG. 5C. During the drive stroke, driver (86B) engages proximal notch (74) and will in unison rotate needle (70) about 180 degrees along an orbital path to its extended position. Needle (70) will span across arms (93A, 93B) between exit port (97A) and entrance port (97B). Tissue interposed between arms (93A, 93B) will be pierced by leading end (71) of needle (70).

When first user input member (12) (see FIG. 1) is released and the spring return opens the trigger, needle driver (86) reciprocates through its return stroke where it orbits along an angular range of motion about 180 degrees clockwise back to the return position shown in FIG. 5A. During the return stroke, driver (86B) slides over the needle (70). Driver (86B) is then adjacent the distal notch (74). When first user input member (12) is depressed again closing the trigger, needle driver (86) will again be actuated through its drive stroke where it orbits along an angular range of motion about 180 degrees counterclockwise to the driven position as shown in FIG. 5C. During the drive stroke, driver (86B) engages distal notch (74) and will in unison drive needle (70) orbitally along an angular range of motion about 180 degrees back to its retracted position. Suture (73) (see FIG. 3A) will follow needle (70) and be threaded through the pierced tissue.

When first user input member (12) (see FIG. 1) is again released and the spring return opens the trigger, needle driver (86) again reciprocates through its return stroke where it orbits along an angular range of motion about 180 degrees clockwise back to its returned position as shown in FIG. 5A. During the return stroke, driver (86B) slides over needle (70). Thus, needle (70) is driven in a complete circular path spanning an angular range of 360° in response to first user input member (12) being actuated twice. The sequence may be repeated as needed by the surgeon to achieve the desired suturing task.

Further details, explanations, examples, and alternative embodiments of surgical suturing devices and subcomponents of the foregoing are disclosed in U.S. Pat. No. 9,357,998, entitled “Circular Needle Applier with Articulating and Rotating Shaft,” issued Jun. 7, 2016, the disclosure of which is incorporated by reference herein; U.S. Pat. No. 9,474,522, entitled “Jawed Cartridge Receiving Assembly for Needle Cartridge,” issued Oct. 25, 2016, the disclosure of which is incorporated by reference herein; U.S. Pat. No. 9,375,212, entitled “Circular Needle Applier with Cleats,” issued Jun. 28, 2016, the disclosure of which is incorporated by reference herein; and U.S. Pat. Pub. No. 2016/0367243, entitled “Suturing Instrument with Motorized Needle Drive,” published Dec. 22, 2016, the disclosure of which is incorporated by reference herein. It should be understood that such details, explanations, examples, and alternative embodiments may be readily applied to the above-described instrument (10) and subcomponents thereof.

### II. A Second Exemplary Needle Cartridge Needle Barrier Abutments

In some instances, it may be desirable for the orbital needle path of the suture cartridge to be sized wide enough to slidably receive sutures of varying dimensions and profiles to minimize instances of the suture being immovably constricted therein. For instance, various suturing procedures may require the use of a particularly sized suture to treat the target size. In some instances, the required suture has a greater diameter and/or cross-sectional profile relative to other suitable sutures. Inserting a suture with a relatively greater diameter into the orbital needle path of the suture cartridge may result in the suture becoming jammed. Accordingly, providing a suture cartridge with an enhanced travel path or needle gap that is sized to accommodate various suitably sized sutures may be beneficial to ensure the suture will freely translate along the needle gap without encountering any restrictive impediments. However, enhancing the size of the needle gap may result in the needle received therein to prematurely exit the needle gap due to the comparatively smaller size of the needle relative to the orbital path. Accordingly, it may further be desirable to include one or more needle barriers along and adjacent to the needle gap of the suture cartridge to thereby retain the arcuate needle within the needle gap while the needle and the suture translate through the enhanced orbital path.

The following description provides various examples of needle barriers for a second exemplary needle cartridge (130). Such needle barriers, may include as an illustrative example, one or more retention abutments (200A-D) and/or one or more spaced cutouts (202A-D). Such retention abutments (200A-D) and spaced cutouts (202A-D) described below may be used with any surgical suturing instrument described above and below, and in any of the various procedures described in the various patent references cited herein. To this end, like numbers below indicate like features described above, with 100 series features generally corresponding to features described above. Except as otherwise described below, retention abutments (200A-D) and spaced cutouts (202A-D) described below may be constructed and operable with needle cartridge (30) and instrument (2) described above. Certain details of needle barriers and associated needle cartridge (30) and surgical suturing instrument (2) will therefore be omitted from the following description, it being understood that such details are already provided above in the description of needle cartridge (30) and instrument (2). Other suitable ways in which various surgical suturing instruments may be used will be apparent to those of ordinary skill in the art in view of the teachings herein.

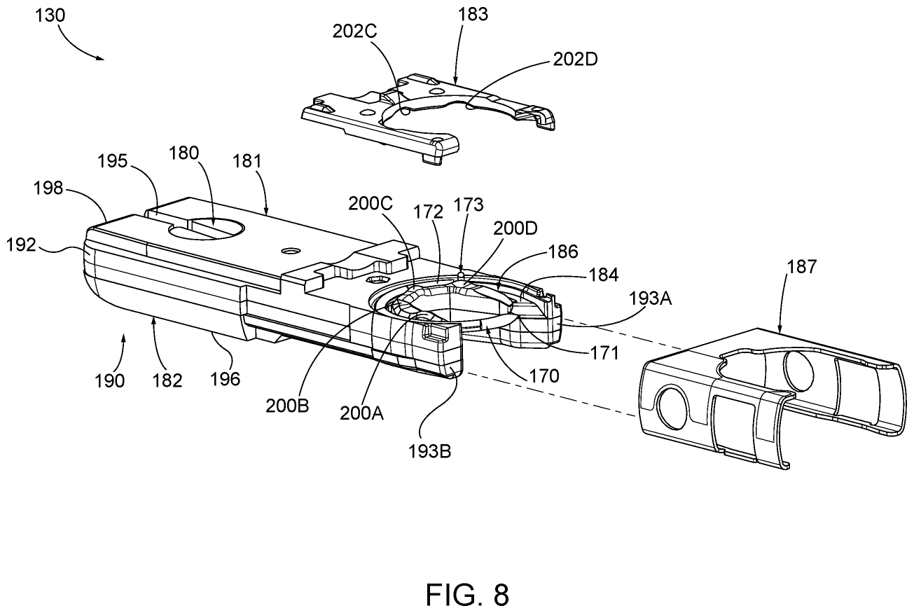

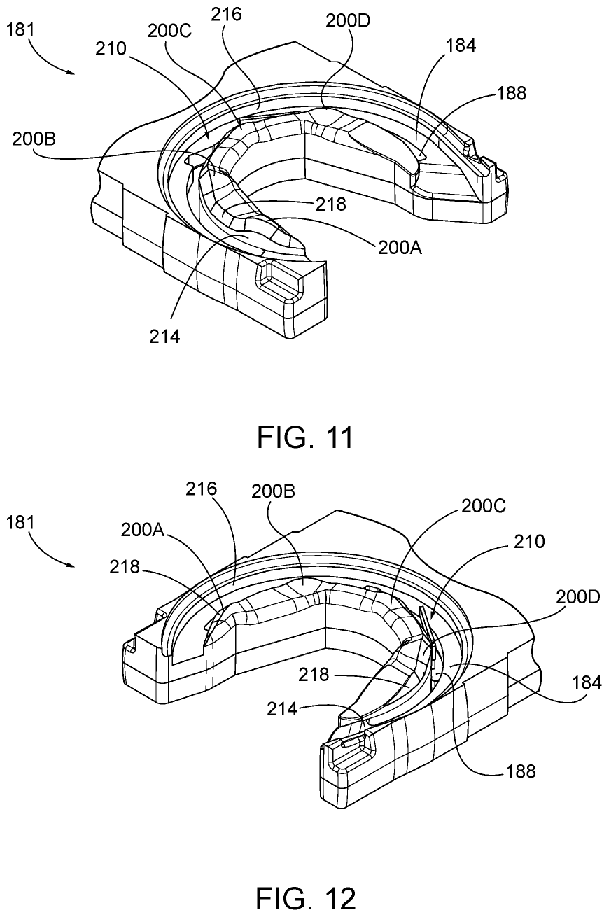

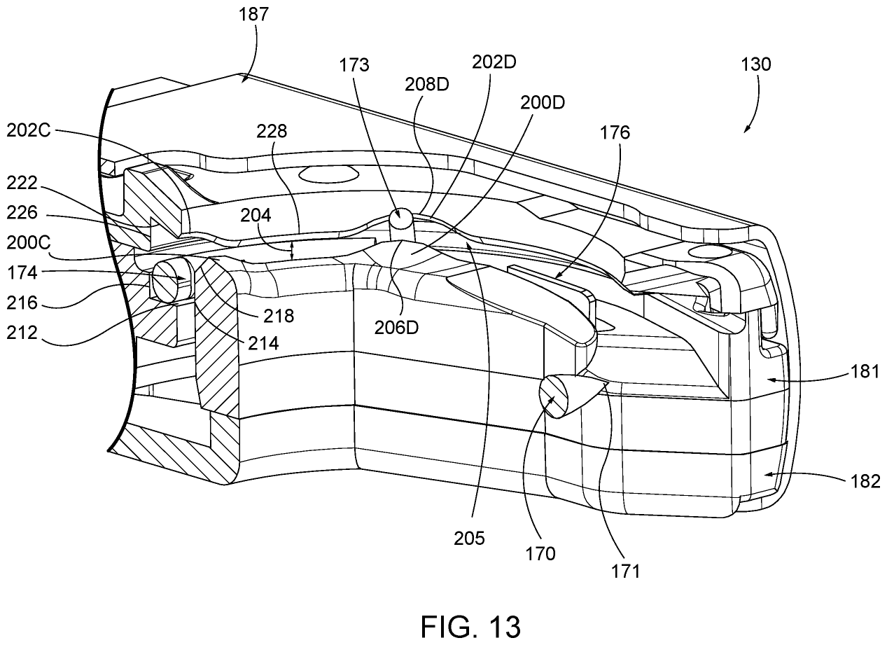

FIGS. 7-16 show a second exemplary needle cartridge (130). As shown in FIGS. 7 and 8, needle cartridge (130) of the present example comprises an arcuate needle (170) having a leading end (171)a trailing end (172) with a length of suture thread (173) extending from trailing end (172) and a proximal notch (174). Needle cartridge (130) further includes a drive assembly (180), a lower body (181), an upper body (182), a needle cover (183), a needle track (184), a needle driver (186), a cage (187), and a carrier track (188). Case (187) is positioned on a disposable cartridge body (190), which has a lower face (191), a pair of longitudinal notched shoulders (192), arms (193A-B), a slot (195), an upper face (196), and a proximal face (198). While some components discussed above with respect to needle cartridge (30) of FIGS. 3A-6 are not shown below in FIGS. 7-16D, these components are readily understood, and may include link (85), rotary input (95), and step (99), among others.

FIGS. 7 and 8 show perspective views of the needle cartridge (130) including some of its various components. For example, needle track (184) extends through at least a portion of cartridge body (190) and defines a circular path. Arcuate needle (170) is movably positioned within needle track (184) and has leading and trailing ends (171, 172). Suture thread (173) extends from trailing end (172) of arcuate needle (170). Needle driver (186) is operatively connected to arcuate needle (170) and lower body (181) of cartridge body (190). Needle driver (186) is configured to orbit arcuate needle (170) along the circular path as described above and shown in greater detail with connection to FIG. 6. Needle cover (183) is secured to cartridge body (190) such that needle cover (183) and cartridge body (190) collectively define a gap (204) therebetween. Gap (204) is configured to receive suture thread (173) therethrough. Gap (204) is large enough to provide sufficient clearance for arcuate needle (170), while small enough to prevent arcuate needle (170) from escaping. While FIG. 8 show sutures (173) terminating proximate to arcuate needle (170), this is merely to more clearly illustrate the needle cartridge (130), and persons skilled in the art would readily understand that suture thread (173) may be any suitable length.

Needle cartridge (130) further includes at least one retention abutment (200A-D), with FIGS. 7 and 8 showing four distinct and separate retention abutments (200A-D). However, less retention abutments (such as one, two, or three retention abutments) as well as more retention abutments (such as five, six, or seven retention abutments) are also envisioned. Retention abutments (200A-D) extend from at least one of cartridge body (190) or from needle cover (183) into gap (204), and, in the present example extend from each of cartridge body (190) and needle cover (183). As shown, needle cover (183), cartridge body (190), and retention abutments (200A-D) at least partially define a tortuous path (205) configured to retain orbiting arcuate needle (170) in needle track (184) while suture thread (173) passes therethrough.

Tortuous path (205) of the present example if further defined by at least one of cartridge body (190) or needle cover (183) spaced cutouts (202A-D) positioned adjacent to gap (204) and extending through cartridge body (190) and needle cover (183). More particularly, with respect to FIGS. 8-13 needle cartridge (130) has four distinct and separate spaced cutouts (202A-D). However, less spaced cutouts (such as one, two, or three spaced cutouts) as well as more spaced cutouts (such as five, six, or seven spaced cutouts) are also envisioned. Spaced cutouts (202A-D) are positioned opposite from retention abutments (200A-D) about gap (204). In other words, gap (204) separates retention abutments (200A-D) from spaced cutouts (202A-D). More specifically, spaced cutouts (202A, 202B, 202C, 202D) are respectively positioned opposite from retention abutments (200A, 200B, 200C, 200D) about gap (204) defining tortuous path (205).

As generally shown in FIGS. 7-15, spaced cutouts (202A-D) are disposed in needle cover (183). Similarly, retention abutments (200A-D) are disposed on lower body (181) of cartridge body (190). However, this orientation may be reversed such that retention abutments (200A-D) are disposed on needle cover (183), and spaced cutouts (202A-D) are disposed in lower body (181) of cartridge body (190). Alternatively, while not shown, lower body (181) of cartridge body (190) may include both retention abutments (200A-D) and spaced cutouts (202A-D) and/or needle cover (183) may include both retention abutments (200A-D) and spaced cutouts (202A-D). As shown, retention abutments (200A-D) are integrally formed together as a unitary piece together with lower body (181) of cartridge body (190). Similarly, spaced cutouts (202A-D) are integrally formed together as a unitary piece together with needle cover (183). However, retention abutments (200A-D) may be separately attached, if desired.

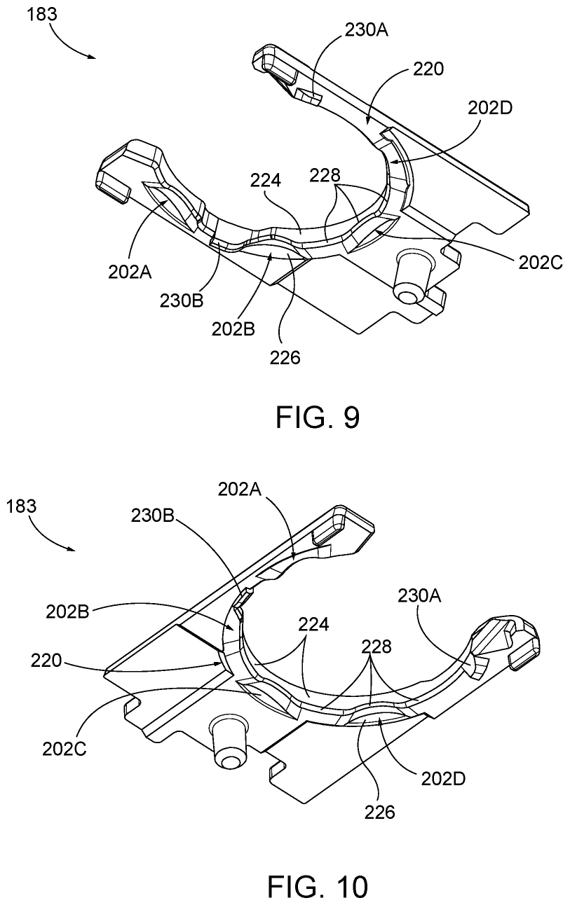

Retention abutments (200A, 200C, 200D) are complementary in shape and size to spaced cutouts (202A, 202C, 202D). More specifically, retention abutment (200A) is complementary in shape and size to spaced cutout (202A). Likewise, retention abutment (200C) is complementary in shape and size to spaced cutout (202C), and retention abutment (200D) is complementary in shape and size to spaced cutout (202D). This results in gap (204) between respective retention abutments (200A, 200C, 200D) and spaced cutouts (202A, 202C, 202D) being generally uniform in distance around needle track (184). More specifically, peaks (206A-D) of retention abutments (200A-D) generally align with recesses (208A-D) of spaced cutouts (202A-D). As shown more particularly in FIG. 9, spaced cutout (202B) has a slightly modified shape that is not entirely complementary to retention member (200B). As a result, gap (204) varies when measured between retention member (200B) and spaced cutout (202B).

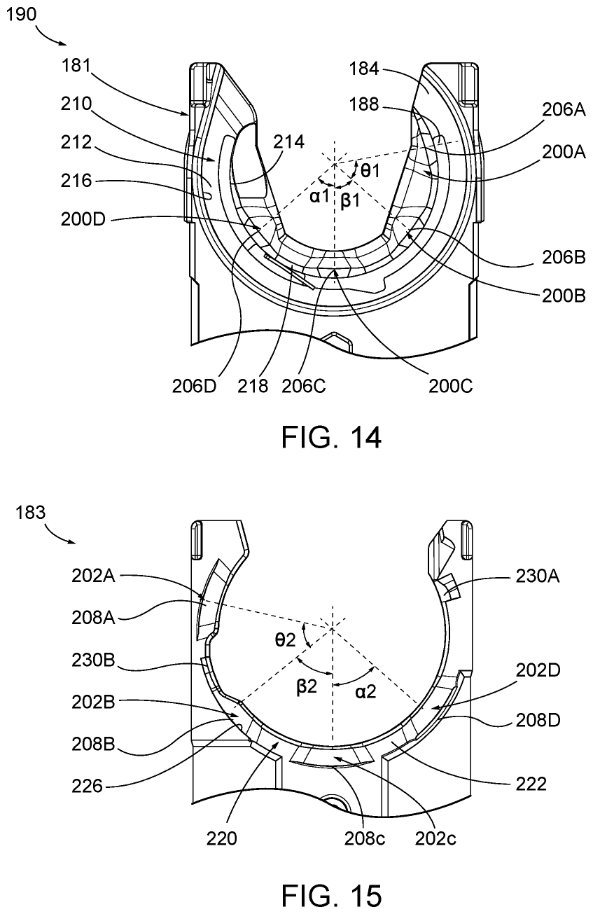

FIG. 14 shows lower body (181) of cartridge body (190) including a track portion (210) that forms part of needle track (184). Track portion (210) includes a base surface (212), a radially inner surface (214) that extends from base surface (212) towards an axis of rotation of arcuate needle (170), and a radially outer surface (216) that extends from base surface (212) away from the axis of rotation of arcuate needle (170). Radially inner surface (214) has a smaller radius of curvature than radially outer surface (216) of cartridge body (190). Radially inner surface (214) of cartridge body (190) includes retention abutments (200A-D). Radially inner surface (214) of lower body (181) of cartridge body (190) includes a tapered portion (218) that is tapered away from base surface (212) and toward gap (204).

FIG. 14 further shows retention abutment (200B) and retention abutment (200C) separated by an angle alpha (“α1”) measured from the center of retention abutment (200B) and retention abutment (200C). Similarly, retention abutment (200C) and retention abutment (200D) are separated by an angle beta (“β1”) measured from the center of retention abutment (200C) to the center of retention abutment (200D). Angle alpha (“α1”) is approximately equal to angle beta (“β1”). Additionally, retention abutment (200A) and retention abutment (200B) are separated by an angle alpha (“θ1”) measured from the center of retention abutment (200A) and retention abutment (200B).

FIG. 15 shows needle cover (183) as including a track portion (220) that forms part of needle track (184). Track portion (220) includes a base surface (222), a radially inner surface (224) that extends from base surface (222) towards an axis of rotation of arcuate needle (170), and a radially outer surface (226) that extends from base surface (222) away from the axis of rotation of arcuate needle (170). Radially inner surface (224) has a smaller radius of curvature than radially outer surface (226) of needle cover (183). Radially inner surface (226) of needle cover (183) includes spaced cutouts (202A-D). Spaced cutouts (202A-D) are deeper adjacent radially outer surface (226) of needle cover (183) than adjacent radially inner surface (224) of needle cover (183). Gap (204) is at least partially defined by radially inner surface (214) of lower body (181) and radially inner surface (224) of needle cover (183). Radially inner surface (214) and radially inner surface (224) each include a respective tapered portion (218, 228) that is tapered toward gap (204).

FIG. 15 further shows spaced cutout (202B) and spaced cutout (202C) separated by an angle alpha (“α2”) measured from the center of spaced cutout (202B) and spaced cutout (202C). Similarly, spaced cutout (202C) and spaced cutout (202D) are separated by an angle beta (“β”) measured from the center of spaced cutout (202C) to the center of spaced cutout (202D). Angle alpha (“α2”) is approximately equal to angle beta (“β2”). Additionally. spaced cutout (202A) and spaced cutout (202B) are separated by an angle alpha (“θ2”) measured from the center of spaced cutout (202A) and spaced cutout (202B).

In addition, FIG. 15 shows needle cover (183) also includes cleats (230A-B) that have multiple advantages compared to other techniques to prevent backward arcuate needle (170) rotation, such as leaf springs or pawls. For example, cleats (230A-B) may be monolithically formed with needle cover (183), thus eliminating separate components to advantageously reduce costs and simplify assembly. For instance, the cover and cleats can be injection molded using materials like polycarbonate, polyetherimide, and the like. In another example, cleats (230A-B) are static thus eliminating moving parts. Cleats (230A-B) project inwardly and dimensionally interfere with arcuate needle (170) in the needle track (184). Each cleat (230A-B) has a ramped leading face allowing arcuate needle (170) to pass under and deflect the cleat (136A, B) outward during the drive strokes. At the end of the drive stroke the stepped trailing end will rotate past one of the cleats (230A-B) that will then deflect inward. The trailing face will engage with the trailing end (172) to prevent backward rotation of arcuate needle (170). Resiliency in the system allows cleats (230A-B) to deflect and return. Further details, explanations, examples, and alternative embodiments of cleats (230A-B) are disclosed in U.S. Pat. No. 9,375,212, entitled “Circular Needle Applier with Cleats,” issued Jun. 28, 2016, the disclosure of which is incorporated by reference herein.

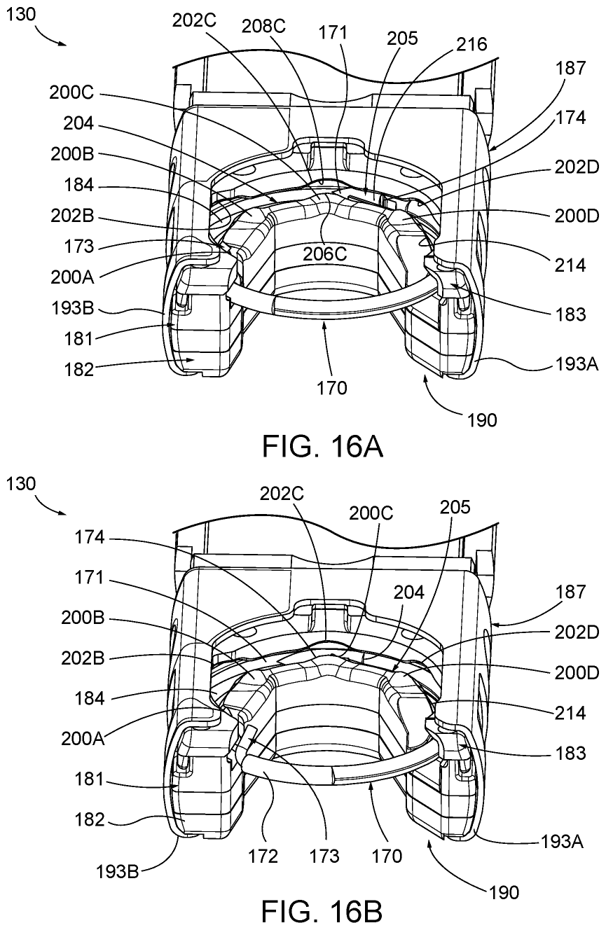

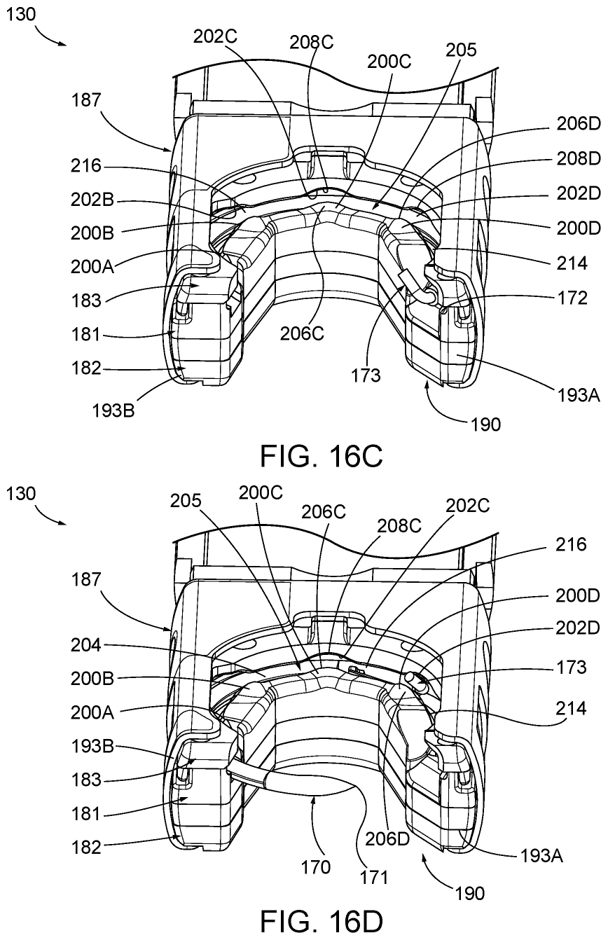

FIGS. 16A-D, show various perspective views of arcuate needle (170) being rotated or orbiting around needle track (184). More specifically, FIG. 14A shows arcuate needle (170) in a first position, FIG. 14B shows arcuate needle (170) in a second position, FIG. 14C shows arcuate needle (170) in a third position, and FIG. 14D shows arcuate needle (170) in a fourth position. As shown, suture thread (173) flexes when traveling past retention abutments (200A-D). As shown, gap (204) is less than the diameter of arcuate needle (170) and is configured to prevent arcuate needle (170) from escaping from needle track (184) when arcuate needle (170) is rotated along the circular path. Gap (204) is designed be large enough to fit arcuate needle (170) and accompanying suture thread (173), but not so large to allow arcuate needle (170) to exit out of needle cartridge (130) under normal use conditions even with component variation, including tolerance stacks.

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A needle cartridge for a surgical suturing instrument, the needle cartridge comprising: (a) a cartridge body; (b) a track extending through at least a portion of the cartridge body and defining a circular path; (c) an arcuate needle movably positioned within the track and having a leading end and a trailing end; (d) a suture thread extending from the trailing end; (e) a needle driver operatively connected to the cartridge body and the arcuate needle, wherein the needle driver is configured to orbit the arcuate needle along the circular path; (f) a needle cover secured to the cartridge body such that the needle cover and the cartridge body define a gap therebetween, wherein the gap is configured to receive the suture thread therethrough; and (g) a first retention abutment extending from at least one of the cartridge body or the needle cover into the gap such that the needle cover, the cartridge body, and the first retention abutment at least partially define a tortuous path configured to retain the orbiting arcuate needle in the track while the suture thread passes therethrough.

### Example 2

The needle cartridge of Example 1, wherein at least one of the cartridge body or the needle cover includes a first spaced cutout positioned adjacent to the gap and further defining the tortuous path, and wherein the first spaced cutout is positioned opposite from the first retention abutment about the gap.

### Example 3

The needle cartridge of Example 2, wherein the first retention abutment is complementary in shape and size to the first spaced cutout.

### Example 4

The needle cartridge of Example 2, wherein the cartridge body includes the first retention abutment and the needle cover includes the first spaced cutout.

### Example 5

The needle cartridge of any one or more of Examples 1 through 4, further comprising: (a) a second retention abutment extending from at least one of the cartridge body or the needle cover into the gap, and (b) a third retention abutment extending from at least one of the cartridge body or the needle cover into the gap, wherein the needle cover, the cartridge body, the first retention abutment, the second retention abutment, and the third retention abutment at least partially define the tortuous path configured to retain the orbiting arcuate needle in the track while the suture thread passes therethrough.

### Example 6

The needle cartridge of Example 5, wherein the first retention abutment and the second retention abutment are separated by a first angle, wherein the second retention abutment and the third retention abutment are separated by a second angle, and wherein the first angle is approximately equal to the second angle.

### Example 7

The needle cartridge of any one or more of Examples 5 through 6, wherein at least one of the cartridge body or the needle cover includes a second spaced cutout and a third spaced cutout that are positioned adjacent to the gap and further defining the tortuous path, wherein the second spaced cutout is positioned opposite from the second retention abutment about the gap, and wherein the third spaced cutout is positioned opposite from the third retention abutment about the gap.

### Example 8

The needle cartridge of Example 7, wherein the first retention abutment is complementary in shape and size to the first spaced cutout, wherein the second retention abutment is complementary in shape and size to the second spaced cutout, and wherein the third retention abutment is complementary in shape and size to the third spaced cutout.

### Example 9

The needle cartridge of any one or more of Examples 5 through 8, wherein the cartridge body includes the first, second, and third retention abutments and the needle cover includes the first, second, and third spaced cutouts.

### Example 10

The needle cartridge of Example 9, wherein the first, second, and third retention abutments are integrally formed together as a unitary piece together with the cartridge body, and wherein the first, second, and third spaced cutouts are integrally formed together as a unitary piece together with the needle cover.

### Example 11

The needle cartridge of any one or more of Examples 9 through 10, wherein the gap is at least partially defined by a radially inner lower surface of the first, second, and third retention abutments and a radially inner upper surface of the first, second, and third spaced cutouts, and wherein the gap is large enough to provide sufficient clearance from the arcuate needle, while small enough to prevent the needle from escaping.

### Example 12

The needle cartridge of Example 11, wherein the radially inner lower surface and the radially inner upper surface each include a tapered portion that is tapered toward the gap.

### Example 13

The needle cartridge of any one or more of Examples 7 through 12, wherein the cartridge body includes a track portion that forms part of the track, wherein the track portion includes a base surface, a radially inner surface that extends from the base surface towards an axis of rotation of the arcuate needle, and a radially outer surface that extends from the base surface away from the axis of rotation of the arcuate needle, wherein the radially inner surface has a smaller radius of curvature than the radially outer surface, wherein the needle cover includes a track portion that forms part of the track, wherein the track portion includes a base surface, a radially inner surface that extends from the base surface towards an axis of rotation of the arcuate needle, and a radially outer surface that extends from the base surface away from the axis of rotation of the arcuate needle, wherein the radially inner surface has a smaller radius of curvature than the radially outer surface of the needle cover, wherein the inner surface of the cartridge body includes the first, second, and third retention abutments and the inner surface of the needle cover includes the first, second and third spaced cutouts, and wherein the inner surface of the cartridge body is tapered away from the base surface and toward the gap.

### Example 14

The needle cartridge of Example 13, wherein each of the first, second, and third spaced cutouts are deeper adjacent the radially outer surface of the needle cover than adjacent the radially inner surface of the needle cover.

### Example 15

The needle cartridge of any one or more of Examples 1 through 4, further comprising: (a) a second retention abutment, a third retention abutment, and a fourth retention abutment disposed on the cartridge body; and (b) a second spaced cutout, a third spaced cutout, and a fourth spaced cutout disposed in the needle cover.

### Example 16

A surgical suturing instrument comprising: (a) a body; (b) a shaft assembly extending distally from the body; (c) a cartridge receiving assembly disposed at a distal end portion of the shaft assembly; and (d) a needle cartridge configured to be coupled with the cartridge receiving assembly, the needle cartridge comprising: (i) a cartridge body, (i) a track extending through at least a portion of the cartridge body and defining a circular path, (iii) an arcuate needle movably positioned within the track and having a leading end and a trailing end, (iv) a suture thread extending from the trailing end, (v) a needle driver operatively connected to the cartridge body and the arcuate needle, wherein the needle driver is configured to orbit the arcuate needle along the circular path, (vi) a needle cover secured to the cartridge body such that the needle cover and the cartridge body define a gap therebetween, wherein the gap is configured to receive the suture thread therethrough, and (vii) a first retention abutment extending from at least one of the cartridge body or the needle cover into the gap such that the needle cover, the cartridge body, and the first retention abutment at least partially define a tortuous path configured to retain the orbiting arcuate needle in the track while the suture thread passes therethrough.

### Example 17

The surgical suturing instrument of Example 16, wherein the needle cartridge further includes a second retention abutment and a third retention abutment, wherein the second retention abutment extends from at least one of the cartridge body or the needle cover into the gap, wherein the third retention abutment extends from at least one of the cartridge body or the needle cover into the gap, and wherein the needle body, the cartridge body, the first retention abutment, the second retention abutment, and the third retention abutment at least partially define the tortuous path configured to retain the orbiting arcuate needle in the track while the suture thread passes therethrough.

### Example 18

The needle cartridge of Example 17, wherein at least one of the cartridge body or the needle cover includes a first spaced cutout, a second spaced cutout, and a third spaced cutout that are positioned adjacent to the gap and further define the tortuous path, and wherein the first spaced cutout is positioned opposite from the first retention abutment about the gap, wherein the second spaced cutout is positioned opposite from the second retention abutment about the gap, and wherein the third spaced cutout is positioned opposite from the third retention abutment about the gap.

### Example 19

A method for retaining an arcuate needle from escaping a track of a needle cartridge operatively coupled with a surgical suturing instrument, wherein the needle cartridge includes a cartridge body, a track extending through at least a portion of the cartridge body and defining a circular path, an arcuate needle movably positioned within the track and having a leading end and a trailing end, a suture thread extending from the trailing end, a needle driver operatively connected to the cartridge body and the arcuate needle, wherein the needle driver is configured to orbit the arcuate needle along the circular path, and a needle cover secured to the cartridge body such that the needle cover and the cartridge body define a gap therebetween, wherein the gap is configured to receive the suture thread therethrough, the method comprising: (a) driving the arcuate needle along the circular path within the track; (b) passing the suture thread through the gap as the needle is driven along the circular path; and (c) retaining the arcuate needle within the track using a first retention abutment extending from at least one of the cartridge body or the needle cover into the gap such that the needle cover, the cartridge body, and the first retention abutment at least partially define a tortuous path configured to retain the orbiting arcuate needle in the track while the suture thread passes therethrough.

### Example 20

The method of Example 19, wherein at least one of the cartridge body or the needle cover includes a first spaced cutout positioned adjacent to the gap, and wherein the first spaced cutout is positioned opposite from the first retention abutment about the gap, and wherein the method further comprises retaining the arcuate needle within the track using the first retention abutment and the first spaced cutout that together at least partially define a tortuous path configured to retain the orbiting arcuate needle in the track while the suture thread passes therethrough.

### IV. Miscellaneous

It should also be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein, is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A needle cartridge for a surgical suturing instrument, the needle cartridge comprising:
(a) a cartridge body;
(b) a track extending through at least a portion of the cartridge body and defining a circular path;
(c) an arcuate needle movably positioned within the track and having a leading end and a trailing end;
(d) a suture thread extending from the trailing end;
(e) a needle driver operatively connected to the cartridge body and the arcuate needle, wherein the needle driver is configured to orbit the arcuate needle along the circular path;
(f) a needle cover secured to the cartridge body such that the needle cover and the cartridge body define a gap therebetween, wherein the gap is configured to receive the suture thread therethrough; and
(g) a first retention abutment extending from at least one of the cartridge body or the needle cover into the gap such that the needle cover, the cartridge body, and the first retention abutment at least partially define a tortuous path configured to retain the orbiting arcuate needle in the track while the suture thread passes therethrough.

2. The needle cartridge of claim 1, wherein at least one of the cartridge body or the needle cover includes a first spaced cutout positioned adjacent to the gap and further defining the tortuous path, and wherein the first spaced cutout is positioned opposite from the first retention abutment about the gap.

3. The needle cartridge of claim 2, wherein the first retention abutment is complementary in shape and size to the first spaced cutout.

4. The needle cartridge of claim 2, wherein the cartridge body includes the first retention abutment and the needle cover includes the first spaced cutout.

5. The needle cartridge of claim 1, further comprising:
(a) a second retention abutment extending from at least one of the cartridge body or the needle cover into the gap, and
(b) a third retention abutment extending from at least one of the cartridge body or the needle cover into the gap, wherein the needle cover, the cartridge body, the first retention abutment, the second retention abutment, and the third retention abutment at least partially define the tortuous path configured to retain the orbiting arcuate needle in the track while the suture thread passes therethrough.

6. The needle cartridge of claim 5, wherein the first retention abutment and the second retention abutment are separated by a first angle, wherein the second retention abutment and the third retention abutment are separated by a second angle, and wherein the first angle is approximately equal to the second angle.

7. The needle cartridge of claim 5, wherein at least one of the cartridge body or the needle cover includes a second spaced cutout and a third spaced cutout that are positioned adjacent to the gap and further defining the tortuous path, wherein the second spaced cutout is positioned opposite from the second retention abutment about the gap, and wherein the third spaced cutout is positioned opposite from the third retention abutment about the gap.

8. The needle cartridge of claim 7, wherein the first retention abutment is complementary in shape and size to the first spaced cutout, wherein the second retention abutment is complementary in shape and size to the second spaced cutout, and wherein the third retention abutment is complementary in shape and size to the third spaced cutout.

9. The needle cartridge of claim 7, wherein the cartridge body includes the first, second, and third retention abutments and the needle cover includes the first, second, and third spaced cutouts.

10. The needle cartridge of claim 9, wherein the first, second, and third retention abutments are integrally formed together as a unitary piece together with the cartridge body, and wherein the first, second, and third spaced cutouts are integrally formed together as a unitary piece together with the needle cover.

11. The needle cartridge of claim 9, wherein the gap is at least partially defined by a radially inner lower surface of the first, second, and third retention abutments and a radially inner upper surface of the first, second, and third spaced cutouts, and wherein the gap is large enough to provide sufficient clearance from the arcuate needle, while small enough to prevent the needle from escaping.

12. The needle cartridge of claim 11, wherein the radially inner lower surface and the radially inner upper surface each include a tapered portion that is tapered toward the gap.

13. The needle cartridge of claim 7, wherein the cartridge body includes a track portion that forms part of the track, wherein the track portion includes a base surface, a radially inner surface that extends from the base surface towards an axis of rotation of the arcuate needle, and a radially outer surface that extends from the base surface away axis of rotation of the arcuate needle, wherein the radially inner surface has a smaller radius of curvature than the radially outer surface,
wherein the needle cover includes a track portion that forms part of the track, wherein the track portion includes a base surface, a radially inner surface that extends from the base surface towards an axis of rotation of the arcuate needle, and a radially outer surface that extends from the base surface away axis of rotation of the arcuate needle, wherein the radially inner surface has a smaller radius of curvature than the radially outer surface of the needle cover,
wherein the inner surface of the cartridge body includes the first, second, and third retention abutments and the inner surface of the needle cover includes the first, second and third spaced cutouts, and
wherein the inner surface of the cartridge body is tapered away from the base surface and toward the gap.

14. The needle cartridge of claim 13, wherein each of the first, second, and third spaced cutouts are deeper adjacent the radially outer surface of the needle cover than adjacent the radially inner surface of the needle cover.

15. The needle cartridge of claim 2, further comprising:
(a) a second retention abutment, a third retention abutment, and a fourth retention abutment disposed on the cartridge body; and
(b) a second spaced cutout, a third spaced cutout, and a fourth spaced cutout disposed in the needle cover.

16. A surgical suturing instrument comprising:
(a) a body;
(b) a shaft assembly extending distally from the body;
(c) a cartridge receiving assembly disposed at a distal end portion of the shaft assembly; and
(d) a needle cartridge configured to be coupled with the cartridge receiving assembly, the needle cartridge comprising:
(i) a cartridge body,
(i) a track extending through at least a portion of the cartridge body and defining a circular path,
(iii) an arcuate needle movably positioned within the track and having a leading end and a trailing end,
(iv) a suture thread extending from the trailing end,
(v) a needle driver operatively connected to the cartridge body and the arcuate needle, wherein the needle driver is configured to orbit the arcuate needle along the circular path,
(vi) a needle cover secured to the cartridge body such that the needle cover and the cartridge body define a gap therebetween, wherein the gap is configured to receive the suture thread therethrough, and
(vii) a first retention abutment extending from at least one of the cartridge body or the needle cover into the gap such that the needle cover, the cartridge body, and the first retention abutment at least partially define a tortuous path configured to retain the orbiting arcuate needle in the track while the suture thread passes therethrough.

17. The surgical suturing instrument of claim 16, wherein the needle cartridge further includes a second retention abutment and a third retention abutment, wherein the second retention abutment extends from at least one of the cartridge body or the needle cover into the gap, wherein the third retention abutment extends from at least one of the cartridge body or the needle cover into the gap, and wherein the needle body, the cartridge body, the first retention abutment, the second retention abutment, and the third retention abutment at least partially define the tortuous path configured to retain the orbiting arcuate needle in the track while the suture thread passes therethrough.

18. The needle cartridge of claim 17, wherein at least one of the cartridge body or the needle cover includes a first spaced cutout, a second spaced cutout, and a third spaced cutout that are positioned adjacent to the gap and further define the tortuous path, and wherein the first spaced cutout is positioned opposite from the first retention abutment about the gap, wherein the second spaced cutout is positioned opposite from the second retention abutment about the gap, and wherein the third spaced cutout is positioned opposite from the third retention abutment about the gap.

19. A method for retaining an arcuate needle from escaping a track of a needle cartridge operatively coupled with a surgical suturing instrument, wherein the needle cartridge includes a cartridge body, a track extending through at least a portion of the cartridge body and defining a circular path, an arcuate needle movably positioned within the track and having a leading end and a trailing end, a suture thread extending from the trailing end, a needle driver operatively connected to the cartridge body and the arcuate needle, wherein the needle driver is configured to orbit the arcuate needle along the circular path, and a needle cover secured to the cartridge body such that the needle cover and the cartridge body define a gap therebetween, wherein the gap is configured to receive the suture thread therethrough, the method comprising:
(a) driving the arcuate needle along the circular path within the track;
(b) passing the suture thread through the gap as the needle is driven along the circular path; and
(c) retaining the arcuate needle within the track using a first retention abutment extending from at least one of the cartridge body or the needle cover into the gap such that the needle cover, the cartridge body, and the first retention abutment at least partially define a tortuous path configured to retain the orbiting arcuate needle in the track while the suture thread passes therethrough.

20. The method of claim 19, wherein at least one of the cartridge body or the needle cover includes a first spaced cutout positioned adjacent to the gap, and wherein the first spaced cutout is positioned opposite from the first retention abutment about the gap, and wherein the method further comprises retaining the arcuate needle within the track using the first retention abutment and the first spaced cutout that together at least partially define a tortuous path configured to retain the orbiting arcuate needle in the track while the suture thread passes therethrough.
